(19) United States
(12) Patent Application Publication
(10) Pub. No.: US 2021/0015677 A1
(43) Pub. Date: Jan. 21, 2021

(54) AN ABSORBENT DRESSING INCORPORATING PH WOUND CONDITION INDICATION

(71) Applicant: KCI LICENSING, INC., Ban Antonio, TX (US)

(72) Inventors: Christopher B. LOCKE, Bournemouth (GB); Timothy Mark ROBINSON, Blandford Forum (GB)

(21) Appl. No.: 17/040,656

(22) PCT Filed: Mar. 29, 2019
(86) PCT No.: PCT/US19/24842

§ 371 Date: Sep. 23, 2020

Related U.S. Application Data
(60) Provisional application No. 62/650,369, filed on Mar. 30, 2018.

(51) Int. Cl. A61F 13/00; A61M 1/00; A61F 13/02

(52) U.S. Cl. CPC A61F 13/00055; A61M 1/0088; A61M 1/0025; A61F 13/0206; A61F 2013/00604; A61F 13/00042; A61F 13/00046; A61M 2205/3324; A61F 2013/00948; A61F 13/00068

(57) ABSTRACT

The present disclosure describes a wound dressing with one or more integrated pH sensors. The pH sensors can measure the pH at different portions of the wound and dressing. The pH sensors can measure the pH of the wound and dressing at different intervals throughout the wearing of the dressing. The pH sensors can provide real-time feedback during wear time. In addition to providing indications of pH, the pH sensors can provide indications of the wound dressing's level of saturation. The pH sensors can be visual indications that the wound dressing is saturated with fluid and should be changed.

### CROSS-REFERENCE TO RELATED APPLICATIONS

This application claims the benefit of and priority to U.S. Provisional Patent Application No. 62/650,369, filed Mar. 30, 2018, the entire contents of which are incorporated herein by reference.

### BACKGROUND OF THE DISCLOSURE

The pH of a wound and the wound fluid can be used as a measure in determining a wound's state. The pH can be factored together with clinician judgement to make therapeutic decisions about wound care. The pH at the wound site can indicate whether the conditions within the wound are conducive to healing. For example, a slightly acidic or neutral pH in the wound can indicate the lack of a bio-film and bacteria in the wound. Accordingly, a slightly acidic pH can be conducive for wound healing. Conversely, an alkaline pH at the wound site may not be conducive to healing. Currently to measure the pH of a wound, a clinician must undertake additional steps before or during the application of wound dressings.

### SUMMARY OF THE DISCLOSURE

The present disclosure describes a wound dressing with one or more integrated pH sensors or other chemical sensors. The pH sensors can measure the pH at different portions of the wound and dressing. The pH sensors can measure the pH of the wound and dressing at different intervals throughout wear time of the dressing. The pH sensors can provide real-time feedback during wear time of pH and other chemical conditions. In addition to providing indications of pH, the pH sensors can provide indications of the wound dressing's level of saturation. The pH sensors can be visual indications that the wound dressing is saturated with fluid and should be changed.

According to at least one aspect of the disclosure, a wound dressing can include a barrier layer. The barrier layer can include a first environmental-facing side and a first wound-facing side. The wound dressing can include a first wicking layer. The first wicking layer can include a second environmental-facing side and a second wound-facing side. The second environmental-facing side can be coupled with the first wound-facing side. The wound dressing can include a first pH indicator strip. A first portion of the first pH indicator strip can be positioned on the second environmental-facing side and a second portion of the first pH indicator strip can be positioned on the second wound-facing side. The wound dressing can include an absorbent layer. The absorbent layer can include a third environmental-facing side and a third wound-facing side. The third environmental-facing side can be coupled with the second wound-facing side. The wound dressing can include a second wicking layer. The second wicking layer can include a fourth environmental-facing side and a fourth wound-facing side. The fourth environmental-facing side can be coupled with the third wound-facing side. The wound dressing can include a second pH indicator strip. A first portion of the second pH indicator strip can be positioned on the second environmental-facing side and a second portion of the second pH indicator strip can be positioned on the fourth wound-facing side.

The first pH indicator strip and the second pH indicator strip can include at least one of a cellulose filter paper, a microporous hydrophilic film, a woven hydrophilic fiber, a non-woven hydrophilic fiber, or a hydrophilic, non-swelling wicking foam.

The first pH indicator strip and the second pH indicator strip can include a pH reactive dye. The first pH indicator strip and the second pH indicator strip can include a polymer binder configured to reduce a migration of the pH reactive dye.

One of the wound dressing's pH indicator strips can be configured to wick a fluid from a wound site. One of the wound dressing's pH indicator strip can be configured to wick a fluid from at least the absorbent layer. The pH indicator strips can include a trigger indicator that can include a moisture released ink.

The barrier layer can include a first portion have a first vapor permeability and a second portion that can have a second vapor permeability that can be different from the vapor permeability of the first portion. The second portion can be configured to enable fluid to evaporate from the first portion of the first pH indicator strip and the first portion of the second pH indicator strip.

The wound dressing can include a third pH indicator strip. A first portion of the third pH indicator strip can be positioned on the second environmental-facing side and a second portion of the third pH indicator strip can be positioned on the fourth wound-facing side. The wound dressing can include a first dissolvable film that can at least partially encase the second portion of the second pH indicator strip and a second dissolvable film that can at least partially encasing the second portion of the third pH indicator strip.

The first dissolvable film can be configured to dissolve after a first predetermined amount of time and the second dissolvable film can be configured to dissolve after a second predetermined amount of time. The first and second predetermined amounts of time can be different.

The barrier layer can include a polyurethane film. The barrier layer can be liquid impermeable and vapor permeable. The wound dressing can include a silicone contact layer coupled with the third wound-facing side of the second wicking layer.

According to at least one aspect of the disclosure, a kit can include a barrier layer, a wound dressing, and an indicator card. The wound dressing can include a first wicking layer and a second wicking layer. The first wicking layer and the second wicking layer can be separated by an absorbent layer. Each of the wicking layers and the absorbent layer can include an environmental-facing side and a wound-facing side. The wound dressing can include a first pH indicator strip and a second pH indicator strip. The indicator card can include a color legend that can map a plurality of colors to a respective pH value.

The indicator card can be configured to couple with the barrier layer. The kit can include a pressure connector or dressing interface that is configured to couple the wound dressing with a negative pressure source.

According to at least one aspect of the disclosure, a method can include applying a wound dressing to a wound site. The wound dressing can include a first pH indicator strip that can be configured to wick a fluid from a wound-facing side of the wound dressing. The wound dressing can include a second pH indicator strip that can be configured to wick the fluid from an interior portion of the wound dressing. The method can include determining, at a first time point, a color of the first pH indicator strip. The method can include determining, at a second time point after the first-time point, a color of the second pH indicator strip.

The method can include comparing the color of the first pH indicator strip to an indicator card to determine a first approximate pH value. The method can include comparing the color of the second pH indicator strip to the indicator card to determine a second approximate pH value.

The method can include determining, at a third time point, a second color of the first pH indicator strip. The method can include determining, at a fourth time point, a second color of the second pH indicator strip. The method can include selecting the third time point to enable a first portion of the fluid to evaporate from the first pH indicator strip. The method can include selecting the fourth time point to enable a second portion of the fluid to evaporate from the second pH indicator strip.

The method can include selecting the first time point after a portion of a first dissolvable film encasing a portion of the first pH indicator strip dissolved. The method can include selecting the second time point after a portion of a second dissolvable film encasing a portion of the second pH indicator strip dissolved.

The first pH indicator strip and the second pH indicator strip can include at least one of a cellulose filter paper, a microporous hydrophilic film, a woven hydrophilic fiber, a non-woven hydrophilic fiber, or a hydrophilic, non-swelling wicking foam.

The first pH indicator strip and the second pH indicator strip can include a pH reactive dye. The first pH indicator strip and the second pH indicator strip can include a polymer binder configured to reduce a migration of a pH reactive dye.

The method can include selecting the first time point after an activation of a moisture trigger indicator of the first pH indicator strip. The method can include applying a negative pressure to at least a portion of the wound dressing.

### BRIEF DESCRIPTION OF THE DRAWINGS

The accompanying drawings are not intended to be drawn to scale. Like reference numbers and designations in the various drawings indicate like elements. For purposes of clarity, not every component may be labeled in every drawing. In the drawings:

FIG. 1 illustrates a top view of an example dressing.

FIG. 2 illustrates a cross-sectional view of the example dressing.

FIG. 3 illustrated an example indicator card that provides a mapping between the pH sensor's color and measured pH.

FIG. 4 illustrates a flow diagram of an example method to determine the pH at a wound site.

### DETAILED DESCRIPTION

The various concepts introduced above and discussed in greater detail below may be implemented in any of numerous ways, as the described concepts are not limited to any particular manner of implementation. Examples of specific implementations and applications are provided primarily for illustrative purposes.

The present disclosure describes a wound dressing with one or more integrated pH sensors or other chemical sensors. The pH sensors can measure the pH at different portions of the wound and dressing and at different times during wear time. The wound dressing, with the integrated pH sensors, can enable the measurement of pH at the start of therapy (e.g., shortly after the application of the wound dressing), towards the end of the wound dressing wear time, or time points there between to show how the pH in the wound alters during treatment.

In addition to providing indications of pH in the wound, the sensors can also provide an indication of when the wound dressing (or absorbents therein) have become full. The pH sensors can provide real-time (or near real-time) indications of the wound pH by utilizing wicking systems and evaporation to enable the pH sensors to continuously absorb new fluid and provide updates on fluid level and pH during wear time. The wound dressing can also include other sensors that can detect other chemical markers during wear time.

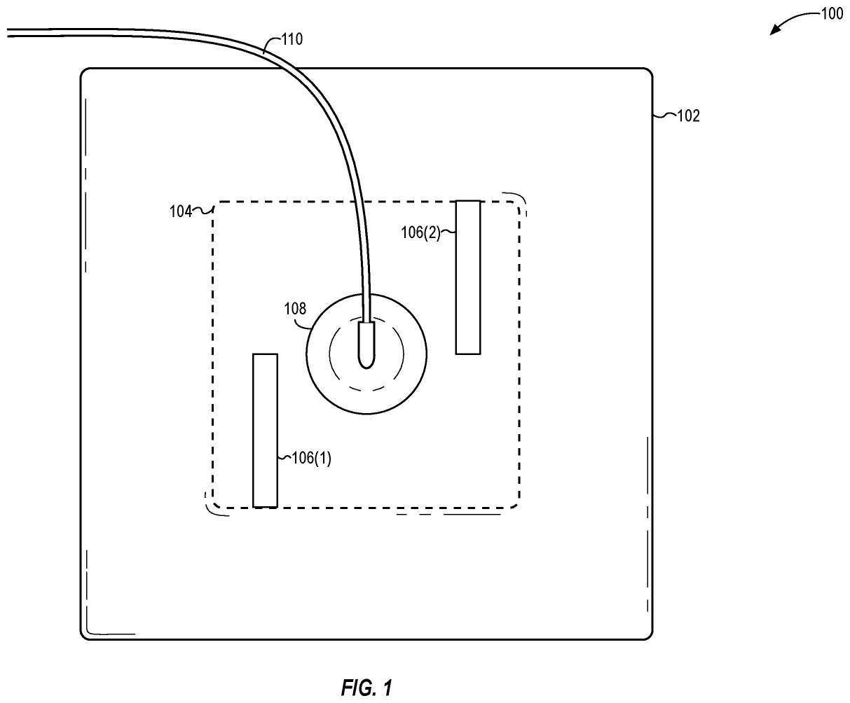

FIG. 1 illustrates a top view of a dressing 100. The dressing 100 includes a barrier layer 102 that covers an absorbent island 104. The absorbent island 104 includes a first sensor 106(1) and a second sensor 106(2), which can generally be referred to as sensors 106. The dressing 100 includes a dressing interface 108 that is coupled with the barrier layer 102 and positioned over the absorbent island 104. The configuration and arrangement of the components of the dressing 100 is provided for illustrative purposes only. The sensors 106 described herein can be incorporated into other forms or types of wound and other dressings. For example, the sensors 106 can be incorporated into wound dressings that include fewer or more internal layers, different internal compositions (e.g., wound dressings including collagen or cellulose-based foams or multi-layered absorbent wound dressings), or different shapes. The sensors 106 can be incorporated into wound dressings that can be used with negative pressure wound therapy systems. The sensors 106 can be incorporated into adhesive foam dressings used to absorb fluid from exuding wounds.

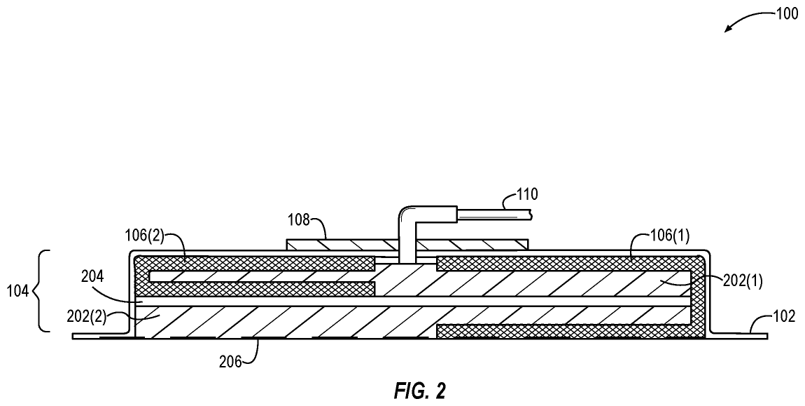

The absorbent island 104 and the sensors 106 are described further in relation to FIG. 2. As an overview, the absorbent island 104 can include one or more layers. For example, the absorbent island 104 can include one or more absorbent layers and one or more fluid wicking layers. The wicking layers can distribute fluid through the absorbent island 104. The fluid can be absorbed and stored by the absorbent layers. The absorbent island 104 can also include one or more sensors 106. The sensors 106 can include a pH reactive dye that changes color based on the pH of the fluid absorbed by the sensor 106. Wicking portions of the sensors 106 can wick fluid from different layers of the absorbent island 104 or from a wound site covered by the absorbent island 104.

The dressing 100 includes the barrier layer 102. The barrier layer 102 can be referred to as an upper drape cover. The barrier layer 102 can extend past the periphery of the absorbent island 104. The wound-facing side of the barrier layer 102 can include an adhesive that can couple the absorbent island 104 with the barrier layer 102. The adhesive on the portion of the barrier layer 102 extending past the perimeter of the absorbent island 104 can couple the dressing 100 with a contact surface, such as the patient's skin surrounding a wound.

The barrier layer 102 can be transparent. For example, the barrier layer 102 can be transparent to enable the sensors 106, positioned below the barrier layer 102, to be viewable to a wearer or healthcare professional. The barrier layer 102 can include portions that are transparent and portions that are not transparent. For example, the barrier layer 102 can include one or more transparent windows that are positioned over the sensors 106 to provide viewable access to the sensors 106. The other portions of the barrier layer 102 can be non-transparent.

The barrier layer 102 can form a fluid seal with the contact surface. The barrier layer 102 can be vapor permeable and liquid impermeable. The barrier layer 102 can include hydrophilic polyurethane, cellulosics, hydrophilic polyamides, polyvinyl alcohol, polyvinyl pyrrolidone, hydrophilic acrylics, hydrophilic silicone elastomers, an INSPIRE 2301 material from Expopack Advanced Coatings of Wrexham, United Kingdom having, for example, natural rubbers, polyisoprene, styrene butadiene rubber, chloroprene rubber, polybutadiene, nitrile rubber, butyl rubber, ethylene propylene rubber, ethylene propylene diene monomer, chlorosulfonated polyethylene, polysulfide rubber, polyurethane (PU), EVA film, co-polyester, silicones, a silicone drape, a 3M Tegaderm® drape, a polyurethane (PU) drape such as one available from Avery Dennison Corporation of Pasadena, Calif., polyether block polyamide copolymer (PEBAX), for example, from Arkema, France, Expopack 2327, or other appropriate material.

The barrier layer 102 can have a thickness between about 5 μm to about 75 μm, between about 10 μm to about 50 μm, between about 10 μm to about 35 μm, or between about 15 μm and about 25 μm.

Different portions of the barrier layer 102 can include different materials. The different materials can be selected to have different liquid and vapor permeability characteristics. For example, a first portion of the barrier layer 102 can have a first level of vapor permeability (or breathability) and a second portion of the barrier layer 102 can have a second level of vapor permeability. The second level of vapor permeability can be greater than the first level of vapor permeability. The second portion of the barrier layer 102 (with the second, higher level of vapor permeability) can be positioned over at least one of the sensors 106. The relatively high level of vapor permeability can enable fluid in the sensor 106 to evaporate. As the fluid evaporates the sensor 106 dries and the sensor 106 can draw additional fluid from the wound (or other portions of the dressing 100). The drying of the sensor 106 and then absorption of additional fluid enables the sensors 106 to provided updated indications of the pH level present at the wound site and in the dressing 100.

The dressing 100 also includes a dressing interface 108. The dressing interface 108 can be positioned in an opening in the barrier layer 102 and in fluidic communication with the absorbent island 104. The dressing interface 108 can include a port to which tubing 110 can be coupled. The tubing 110 can be coupled with a negative pressure source, such as a pump. The pump can draw a vacuum to generate a negative pressure (with respect to the external environmental pressure) at the wound site that is sealed by the barrier layer 102. The dressing interface 108 can include a medical-grade, soft polymer or other pliable material. For example, the dressing interface 108 can include polyurethane, polyethylene, polyvinyl chloride (PVC), fluorosilicone, ethylene-propylene, or DEHP-free PVC.

FIG. 2 illustrates a cross-sectional view of the example dressing 100. As illustrated, the dressing 100 includes the bottom barrier layer 206, which can be referred to as a lower sealing drape or a silicone contact layer. The absorbent island 104 of the dressing 100 can include a first wicking layer 202(1) and a second wicking layer 202(2), which can collectively be referred to as wicking layers 202. The wicking layers 202 can be separated by an absorbent layer 204. The absorbent island 104 can also include the sensors 106.

The absorbent island 104 illustrated in FIG. 2 includes a first sensor 106(1) and a second sensor 106(2). The sensors 106 can be configured as pH indicator strips that can extend from a wound-facing side or internal portion of the dressing 100 to an environmental-facing side of the dressing 100. The sensor 106(1) can be configured to absorb fluid from the wound site. For example, a first end of the sensor 106(1) can be coupled with or positioned against the wound-facing side of the wicking layer 202(2). In another example, the first end of the sensor 106(1) can be positioned within the wicking layer 202(2). A second end of the sensor 106(1) can be positioned on or coupled with an environmental-facing side of the wicking layer 202(1). The second end of the sensor 106(1) can be viewable through the barrier layer 102. The barrier layer 102 can include windows over the sensors 106 or the barrier layer 102 can be transparent to enable the sensors 106 to be viewed through the barrier layer 102.

The sensor 106(2) can absorb fluid from the interior of the absorbent island 104. For example, a first end of the sensor 106(2) can be positioned between an environmental-facing side of the absorbent layer 204 and a wound facing side of the wicking layer 202(1). The first end of the sensor 106(2) can be placed into one of the wicking layers 202 or the absorbent layer 204. The sensor 106(2) (or other sensor 106) can be used to determine when the dressing 100 should be changed. For example, the first end of the sensor 106(2) can be positioned on the environmental facing side of the absorbent layer 204. In this example, the sensor 106(2) may absorb fluid form the absorbent island 104 once the absorbent layer 204 is substantially saturated with fluid. A pH indicating reaction (or activation of a moisture activated ink) at the second end of the sensor 106(2) can begin once the sensor 106(2) absorbs fluid after the saturation of the absorbent layer 204 and indicates to a user that the absorbent island 104 is nearing a fluid saturation level.

The example dressing 100 illustrated in FIGS. 1 and 2 includes two sensors 106. The dressing 100 can include between about 1 and about 10, between about 2 and about 8, between about 2 and about 6, or between about 2 and 4 sensors 106. The portions of the sensors 106 that absorbs fluid from the wound site or absorbent island 104 can each be located at different depths of the absorbent island 104 or one or more of the portions can be positioned at the same depth of the absorbent island 104. For example, two sensors 106 may terminate on the wound-facing side of the wicking layer 202 nearest the wound. One of the two sensors 106 can termination near the perimeter of the absorbent island 104 (and absorb fluid from the perimeter of the wound) and a second of the two sensors 106 can terminate near the center of the absorbent island 104 (and absorb fluid from the center of the wound).

The sensors 106 can include pH reactive dyes that change color to indicate the pH of absorbed fluid. The sensors 106 can be treated with a polymer binder that can reduce the migration of the pH reactive dye when the dye is exposed to a fluid. The pH reactive dye can include a pH dye mixture such as phenolphthalein, methyl red, bromothymol blue, and thymol blue. The pH dye mixture can be printed onto the wicking material to form the sensors 106. The wicking material of the sensors 106 can include at least one of a cellulose filter paper, a microporous hydrophilic film, a woven hydrophilic fiber, a non-woven hydrophilic fiber, or a hydrophilic, non-swelling wicking foam. A polymer binder can also be printed or applied to the sensors 106 to prevent the pH reactive dyes from migrating when exposed to a fluid.

In some implementations, the pH reactive dye can be re-settable. The pH reactive dye can continue to react as fresh fluid is absorbed by the sensor 106. For example, the as fluid evaporates from the sensor 106, the sensor 106 can draw in new fluid to which the pH reactive dye reacts and provides an updated indication of pH at the wound or wound dressing's core.

The portion of the sensors 106 that can be viewed through the barrier layer 102 can include a trigger indicator. The trigger indicator can include a moisture sensitive ink that is released, becomes visible, or changes color in the presence of a fluid. Activation of the trigger indicator can indicate the fluid is present in the sensor 106 and that the sensor 106 is active. Activation of the trigger indicator without a subsequent color change (or other reaction) of the pH indicator strip's pH reactive dye can indicate that the malfunctioned or did not activate properly. In some implementations, activation of the trigger indicator can indicate that the sensor 106 has absorbed enough fluid to for the pH reactive dye to make an accurate measurement of the fluid's pH value.

Portions of the sensors 106 can be wrapped, coated, or encased within a dissolvable film. Once exposed to a fluid, the dissolvable film can dissolve after a predetermined amount of time. For example, the dissolvable film can dissolve after about 30 minutes, about 1 hour, 3 hours, 6 hours, 12 hours, 1 day, or several days after exposure to a fluid. The rate at which the dissolvable film dissolves or degrades in the presence of a fluid can be control by the materials of the dissolvable film and/or by the thickness of the dissolvable film applied to the sensors 106. For example, a first dissolvable film that is about twice a thick as a second dissolvable film can take about twice as long to dissolve.

Before dissolving, the dissolvable film can substantially prevent the sensors 106 from absorbing fluid. The dissolvable film can enable the pH indicator strips to begin reacting to the pH of absorbed fluid after the predetermined amount of time. The rate at which the dissolvable film dissolves introduces a delay, from the placement of the dressing 100, before the sensors 106 begin providing pH readings. For example, the dissolvable film can substantially prevent the sensors 106 from absorbing fluid for one day such that the pH reactive dye of the sensors 106 do not provide readings until one day after the placement of the dressing 100.

Different sensors 106 in an example dressing 100 can include dissolvable films that dissolve at different times (or rates) when exposed to a fluid. For example, a first sensor 106 can include a dissolvable film that dissolves after one day and provides a pH reading one day post dressing placement. A second sensor 106 can include a dissolvable film that dissolves after two days and provides a pH reading two days post dressing placement.

The absorbent island 104 can include one or more wicking layers 202. The wicking layers 202 can be fluidic communication with the absorbent layer 204. The wicking layers 202 can help distribute a fluid to and throughout the absorbent layer 204. The wicking layers 202 can include grain structures that distribute fluid through the wicking layers 202.

The absorbent island 104 can include an absorbent layer 204. The absorbent layer 204 can be laminated between or coupled with wicking layers 202. The absorbent layer 204 can include sodium polyacrylate super absorbers, cellulosics (carboxy methyl cellulose and salts such as sodium CMC), or alginates. In some implementations, the absorbent layer 204 can include a hydrogel or hydrogel composition. Several examples of hydrogels and hydrogel compositions which can be used to the absorbent layer 204 are described in detail in U.S. Pat. No. 8,097,272 issued Jan. 17, 2012, U.S. Pat. No. 8,664,464 issued Mar. 4, 2014, and U.S. Pat. No. 8,058,499 issued Nov. 15, 2011. The entire disclosure of each of these patents is incorporated by reference herein.

The expressions “hydrogel” and “hydrogel compositions” can include any hydrophilic gels and gel compositions. The compositions can include organic non-polymeric components in the absence of water. For example, the absorbent layer 204 can be formed from a polyurethane that entraps water to form a gel. The absorbent layer 204 can be substantially continuous, substantially non-porous, or non-foamed. The absorbent layer 204 can include a flexible plasticized hydrophilic polymer matrix having a substantially continuous internal structure. The density of absorbent layer 204 may be between about 0.5 g/cm3 and about 1.1 g/cm3, between about 0.8 g/cm3 and about 1.1 g/cm3, or between about 0.9 and about 1.1 g/cm3. The thickness of the absorbent layer 204 can be between about 1 mm and about 10 mm, between about 2 mm and about 7 mm, or between about 2 mm and about 5 mm.

In some implementations, the absorbent layer 204 can be cross-linked. The absorbent layer 204 can be substantially insoluble in water at ambient temperatures. The absorbent layer 204 can absorb and entrap liquid to provide a highly hydrated gel structure in contrast to the porous foam structure of foam layer 108. The gel of the absorbent layer 204 can absorb between about 1 g/g and about 10 g/g, between about 2 g/g and about 7 g/g, or between about 2 g/g and about 5 g/g of physiological saline at 20°.

In some implementations, the dry weight of the absorbent layer 204 is from about 1000 g/m2 to about 5000 g/m2 or between about 2000 g/m2 to about 4000 g/m2. In some implementations, the absorbent layer 204 includes between about 1% and about 30%, between about 5% and about 25%, or between about 10% and about 20% by weight of water before use. The absorbent layer 204 can contain between about 1% and about 40%, between about 5% and about 20%, or between about 5% and about 15% by weight one or more humectants. The humectants can include glycerol, propylene glycol, sorbitol, mannitol, polydextrose, sodium pyrrolidine carboxylic acid (NaPCA), hyaluronic acid, aloe, jojoba, lactic acid, urea, gelatin, lecithin, or any combination thereof. The entrapped water and optional humectants can give the hydrogel a soft, moist wound-friendly surface for contacting the wound.

The dressing 100 can include a base barrier layer 206. The base barrier layer 206 may be a soft, pliable material suitable for providing a fluid seal with the tissue site 104 as described herein. For example, the base barrier layer 206 can include a silicone gel, a soft silicone, hydrocolloid, hydrogel, polyurethane gel, polyolefin gel, hydrogenated styrenic copolymer gels, a foamed gel, a soft closed cell foam such as polyurethanes and polyolefins, polyurethane, polyolefin, or hydrogenated styrenic copolymers.

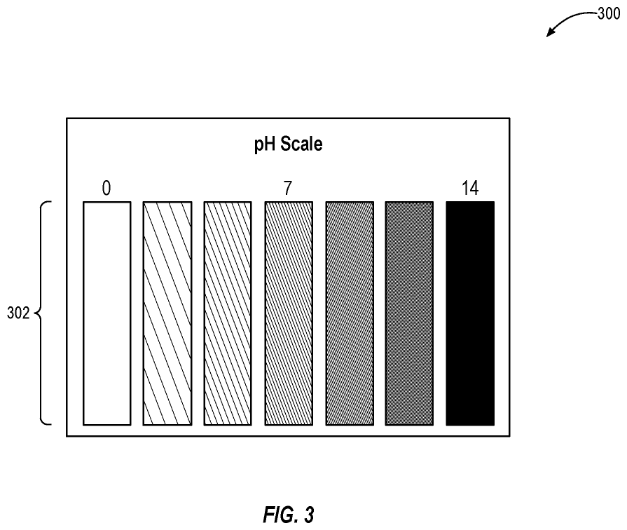

FIG. 3 illustrates an example pH indicator card 300. As described above, as the pH reactive dye in the sensors 106 reacts to absorbed fluid, the pH reactive dye can change color to indicate the pH of the fluid. The card 300 can include legend 302 that associates colors with pH values. The legend 302 can include a separated layout with plurality of individual color blocks (as illustrated in FIG. 3). The legend 302 can include a jointed layout that includes a single color block that includes a continuous spectrum of colors. The card 300 can include numerical values (or ranges) that map different colors of the legend 302. To determine a pH value, the colors of the legend 302 can be compared to the color of the pH indicator strip's pH reactive dye. Once the color is identified, the color to numerical scale mapping can be used to identify the numerical value that corresponds to the color of the pH indicator strip's pH reactive dye.

The card 300 can include be included in a kit with the dressing 100. The card 300 can be a standalone card that the user can use to covert the color of the sensor 106 into a pH value. The card 300 can include an adhesive backing that enables the card 300 to be coupled with the dressing 100 or patient's chart. The card 300 can include labeling areas where the patient's information can be printed or written on the card 300. In some implementations, the legend 302 can be printed directly onto the barrier layer 102 or other component of the dressing 100 rather than being a separate component of the dressing 100.

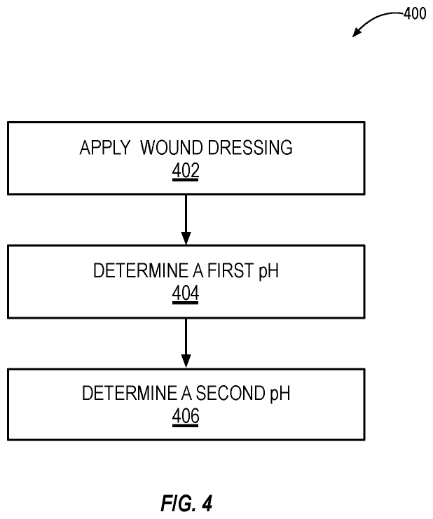

FIG. 4 illustrates a flow diagram of an example method 400 to determine the pH at a wound site. The method 400 can include applying a wound dressing (STEP 402). The method 400 can include determining a first pH value (STEP 404). The method 400 can include determining a second pH value (STEP 406).

As set forth above, the method 400 can include applying a wound dressing (STEP 402). Also, referring to FIGS. 1-3, the wound dressing can be any of the wound dressing described herein. For example, the wound dressing can be the dressing 100. The dressing 100 can include one or more sensors 106. The sensors 106 can wick and absorb fluid from the wound site, areas near the wound site, and the interior of the dressing 100. The sensors 106 can include pH reactive dyes that change color to indicate the pH of the fluid to which they are exposed. The sensors 106 can have portions that absorb fluid at different rates and from different locations to provide a view of how the pH of the wound site changes with time. For example, a portion of the sensors 106 can be encased in a dissolvable film that prevents the sensor 106 from absorbing fluid until at least a portion of the dissolvable film has dissolved. The different sensors 106 of the dressing 100 can include dissolvable films of different thicknesses or manufactured from different materials that expose the different sensors 106 to the fluid at different time points.

The method 400 can include determining a color of a pH indicator strip (STEP 404). The sensors 106 can include pH reactive dyes that change color in the presence of a fluid. The color to which the pH reactive dye changes can indicate the pH of the fluid. Determining the color of the sensor 106 can occur at a first time point. The sensor 106 can include a dissolvable film that prevents the pH indicator strip's pH reactive dye from interacting with the fluid for a predetermined amount of time. The first time point can be at a time after the dissolvable film has dissolved. The first time point can be after a time that the pH reactive dye is exposed to fluid.

The color of the sensor 106 can be compared to the legend indicated on the card 300. The user can find a portion of the legend 302 that is similar in color to the color of the pH indicator strip's pH reactive dye. The legend 302 can include a numerical scale that maps the colors of the legend 302 to different pH values. The legend 302 can be used to map the color of the pH indicator strip's pH reactive dye to a pH value.

The method 400 can include determining, at a second time point, a color of a pH indicator strip (STEP 406). The second time point can be at a time after the time point of STEP 404. In some implementations, the second time point can be at the same time as the time point of STEP 404. For example, the dressing 100 can include different multiple sensors 106 that absorb fluid from different regions of the wound site. A STEP 406, the color can be measured or otherwise determined at the same or a different sensor 106 as the sensor 106 of STEP 404.

For example, to determine a second color of sensor 106 of STEP 404, a portion of the barrier layer 102 above the sensor 106 can have a vapor permeability that enables the fluid in the sensor 106 to evaporate from the sensor 106. As the fluid evaporates, the sensor 106 can absorb additional fluid that reacts to react with pH reactive dye to indicate an updated pH value.

Having now described some illustrative implementations, it is apparent that the foregoing is illustrative and not limiting, having been presented by way of example. In particular, although many of the examples presented herein involve specific combinations of method acts or system elements, those acts and those elements may be combined in other ways to accomplish the same objectives. Acts, elements and features discussed in connection with one implementation are not intended to be excluded from a similar role in other implementations or implementations.

As used herein, the term “about” and “substantially” will be understood by persons of ordinary skill in the art and will vary to some extent depending upon the context in which it is used. If there are uses of the term which are not clear to persons of ordinary skill in the art given the context in which it is used, “about” will mean up to plus or minus 10% of the particular term.

Where technical features in the drawings, detailed description or any claim are followed by reference signs, the reference signs have been included to increase the intelligibility of the drawings, detailed description, and claims. Accordingly, neither the reference signs nor their absence has any limiting effect on the scope of any claim elements.

The systems and methods described herein may be embodied in other specific forms without departing from the characteristics thereof. The foregoing implementations are illustrative rather than limiting of the described systems and methods. Scope of the systems and methods described herein is thus indicated by the appended claims, rather than the foregoing description, and changes that come within the meaning and range of equivalency of the claims are embraced therein.

1. A wound dressing comprising:
a barrier layer comprising a first environmental-facing side and a first wound-facing side;
a first wicking layer comprising a second environmental-facing side and a second wound-facing side, wherein second environmental-facing side is coupled with the first wound-facing side;
a first pH indicator strip, wherein a first portion of the first pH indicator strip is positioned on the second environmental-facing side and a second portion of the first pH indicator strip is positioned on the second wound-facing side;
an absorbent layer comprising a third environmental-facing side and a third wound-facing side, wherein the third environmental-facing side is coupled with the second wound-facing side;
a second wicking layer comprising a fourth environmental-facing side and a fourth wound-facing side, wherein the fourth environmental-facing side is coupled with the third wound-facing side; and
a second pH indicator strip, wherein a first portion of the second pH indicator strip is positioned on the second environmental-facing side and a second portion of the second pH indicator strip is positioned on the fourth wound-facing side.

2. The wound dressing of claim 1, wherein the first pH indicator strip and the second pH indicator strip comprise
at least one of a cellulose filter paper, a microporous hydrophilic film, a woven hydrophilic fiber, a non-woven hydrophilic fiber, or a hydrophilic, non-swelling wicking foam, or
a pH reactive dye, optionally wherein the first pH indicator strip and the second pH indicator strip comprise a polymer binder configured to reduce a migration of the pH reactive dye, or
a trigger indicator comprising a moisture released ink.

3. (canceled)

4. (canceled)

5. The wound dressing of claim 1, wherein the second pH indicator strip is configured to wick a fluid from a wound site.

6. The wound dressing of claim 1, wherein the first pH indicator strip is configured to wick a fluid from at least the absorbent layer.

7. (canceled)

8. The wound dressing of claim 1, wherein the barrier layer comprises a first portion have a first vapor permeability and a second portion having a second vapor permeability different than the first portion, optionally wherein the second portion is configured to enable fluid to evaporate from the first portion of the first pH indicator strip and the first portion of the second pH indicator strip.

9. (canceled)

10. The wound dressing of claim 1, further comprising a third pH indicator strip, wherein a first portion of the third pH indicator strip is positioned on the second environmental-facing side and a second portion of the third pH indicator strip is positioned on the fourth wound-facing side.

11. The wound dressing of claim 10, further comprising a first dissolvable film at least partially encasing the second portion of the second pH indicator strip and a second dissolvable film at least partially encasing the second portion of the third pH indicator strip, optionally wherein the first dissolvable film is configured to dissolve after a first predetermined amount of time and the second dissolvable film is configured to dissolve after a second predetermined amount of time that is different than the first predetermined amount of time.

12. (canceled)

13. The wound dressing of claim 1, wherein the barrier layer comprises a polyurethane film or is liquid impermeable and vapor permeable.

14. (canceled)

15. The wound dressing of claim 1, further comprising a silicone contact layer coupled with the third wound-facing side of the second wicking layer.

16. A kit comprising:
a barrier layer;
a wound dressing comprising:
a first wicking layer comprising a first environmental-facing side and a first wound-facing side;
a first pH indicator strip, wherein a first portion of the first pH indicator strip is positioned on the first environmental-facing side and a second portion of the first pH indicator strip is positioned on the first wound-facing side;
an absorbent layer comprising a second environmental-facing side and a second wound-facing side, wherein the second environmental-facing side is coupled with the first wound-facing side;
a second wicking layer comprising a third environmental-facing side and a third wound-facing side, wherein the third environmental-facing side is coupled with the second wound-facing side; and
a second pH indicator strip, wherein a first portion of the second pH indicator strip is positioned on the first environmental-facing side and a second portion of the second pH indicator strip is positioned on the third wound-facing side; and
an indicator card comprising a plurality of possible colors of the first pH indicator strip and the second pH indicator strip and a respective pH value, optionally wherein the indicator card is configured to couple with the barrier layer.

17. (canceled)

18. The kit of claim 16, further comprising a second wound dressing comprising 45% oxidized regenerated cellulose by weight and 55% collagen by weight or a second wound dressing comprising a polyvinyl alcohol foam.

19. (canceled)

20. The kit of claim 16, further comprising a pressure connector configured to couple with a negative pressure source.

21. A method comprising:
applying a wound dressing to a wound site, the wound dressing comprising:
a first pH indicator strip configured to wick a fluid from a wound-facing side of the wound dressing; and
a second pH indicator strip configured to wick the fluid from an interior portion of the wound dressing;
determining, at a first time point, a color of the first pH indicator strip; and
determining, at a second time point after the first time point, a color of the second pH indicator strip.

22. The method of claim 21, further comprising:
comparing the color of the first pH indicator strip to an indicator card to determine a first approximate pH level; and
comparing the color of the second pH indicator strip to the indicator card to determine a second approximate pH level.

23. The method of claim 21, further comprising:
determining, at a third time point, a second color of the first pH indicator strip; and
determining, at a fourth time point, a second color of the second pH indicator strip.

24. The method of claim 23, further comprising:
selecting the third time point to enable a first portion of the fluid to evaporate from the first pH indicator strip; and
selecting the fourth time point to enable a second portion of the fluid to evaporate from the second pH indicator strip.

25. The method of claim 21, further comprising:
selecting the first time point after a portion of a first dissolvable film encasing a portion of the first pH indicator strip dissolved; and
selecting the second time point after a portion of a second dissolvable film encasing a portion of the second pH indicator strip dissolved.

26. The method of claim 21, wherein the first pH indicator strip and the second pH indicator strip comprise
at least one of a cellulose filter paper, a microporous hydrophilic film, a woven hydrophilic fiber, a non-woven hydrophilic fiber, or a hydrophilic, non-swelling wicking foam, or
a pH reactive dye, or
a polymer binder configured to reduce a migration of a pH reactive dye.

27. (canceled)

28. (canceled)

29. The method of claim 21, further comprising selecting the first time point after an activation of a moisture trigger indicator of the first pH indicator strip.

30. The method of claim 21, further comprising applying a negative pressure to at least a portion of the wound dressing.
